(19) United States
(12) Patent Application Publication
(10) Pub. No.: US 2023/0142943 A1
(43) Pub. Date: May 11, 2023

(54) METHOD FOR TREATING CHRONIC OSTEOMYELITIS OF THE JAWS

(71) Applicant: KUWAIT UNIVERSITY, SAFAT (KW)

(72) Inventors: FAWAZ ALZOUBI, SAFAT (KW); SALEH ALKHALDI, SAFAT (KW)

(21) Appl. No.: 17/520,653

(22) Filed: Nov. 6, 2021

(51) Int. Cl. A61B 17/16; A61B 17/32; A61B 17/3205

(52) U.S. Cl. CPC A61B 17/1673; A61B 17/320068; A61B 17/3205; A61B 2017/00893

(57) ABSTRACT

A method of treating osteomyelitis of the jaws (OMJ) can include sequestrectomy (removing necrotic bone) using piezoelectric vibrations and decortication (creating bleeding spots) using piezoelectric vibrations. The OMJ can include secondary chronic OMJ caused by previous dental implant placements. The method can further include administering dual antibiotic therapy to the patient. The dual antibiotic therapy can include administering Augmentin and Metronidazole to the patient for a period of about three weeks.

### BACKGROUND

### 1. Field

The disclosure of the present patent application relates to medical and surgical methods, and particularly to a method of treating chronic osteomyelitis of the mandible.

### 2. Description of the Related Art

Osteomyelitis is an inflammation or an infection in the bone marrow and/or surrounding bone. The disease may be classified as either acute or chronic, depending on the length of time the infection or symptoms persist. Symptoms may include pain, warmth and/or swelling in the bone. Chronic osteomyelitis may last for years, with slow death of bone tissue from a reduced blood supply. Diabetes, joint replacement, trauma, and injected drug use can lead to osteomyelitis. As people live longer, incidences of osteomyelitis are expected to increase. To complicate matters, an infection, such as following surgery, can occur long after the incision has been closed. An infection buried in a bone can be difficult to detect. The infection is not visible to the eye and taking a culture sample is difficult and painful. Once diagnosed, antibiotics can eliminate many infections. Unfortunately, microorganisms can develop resistances rendering existing antibiotics useless.

Osteomyelitis of the jaws (OMJ) is defined as an inflammatory condition originating from the bone marrow and affecting both the cortical and cancellous bone involving the mandible in the majority of cases possibly due to decreased blood supply. The most common etiological factors are bacterial infections of odontogenic origin represented as pulpal or periodontal infections, trauma, extraction wounds, fractures and possibly foreign bodies such as dental implants.

OMJ can be classified based on its duration as acute or chronic with a time interval of 1 month distinguishing the two classes. Both phases may present with supportive or non-supportive infections leading to compromise in blood supply, causing bony sequestrum due to ischemia and necrosis. Chronic OMJ can be further classified into primary or secondary. Secondary OMJ is characterized by abscess formation, fistula and/or pus discharge with sequestration. Primary OMJ can be described as a non-suppurative chronic inflammation of unknown cause (non-odontogenic). Serious complications, such as fractures and osteolysis, may develop if OMJ is not well managed and treated.

OMJ remains challenging to diagnose, mainly depending on clinical, radiological and pathological findings. Panoramic and/or intra-oral radiographs may aid in diagnosis. Nonetheless, computed tomography is essential to detect periosteal reactions in addition to typical intra-osseosus alterations. Characteristic radiological signs include: irregular radiolucencies (single or multiple), sequestra, moth-eaten appearance with ill-defined cortical borders, bony enlargements, osteosclerotic changes with loss of trabecular pattern, and periosteal reactions resembling an onion peel appearance. Histopathology may be helpful in diagnosis. Findings such as chronic inflammation of varying degrees, heterotopic bone formation, microabcess formation, fibrosis, and hyalinosis are indicative, yet not conclusive, and should be supplemented with clinical and radiological findings.

Although several treatment modalities have been proposed including: antibiotic therapy, sequestrectomy, decortication, botulinum toxin injections, and IV bisphosphonates, there seems to be a lack of agreement or recommended guidelines to manage such cases.

While burs and drills using surgical handpieces are considered effective for cutting hard tissue, there are concerns with using such tools to manage OMJ sites due to possible excessive heat production and induction of further trauma. Furthermore, injury to the soft tissue surrounding the surgical sites is a risk.

Thus, a method of treating osteomyelitis of the jaws solving the aforementioned problems are desired.

### SUMMARY

A method of treating osteomyelitis of the jaws (OMJ) can include using a piezosurgery device for cutting and removing necrotic bone (sequestrectomy) and creating bleeding spots (decortication). The OMJ can include secondary chronic OMJ caused by previous dental implant placements. The method can further include administering dual antibiotic therapy to the patient. The dual antibiotic therapy can include administering Augmentin and Metronidazole to the patient for a period of about three weeks.

These and other features of the present disclosure will become readily apparent upon further review of the following specification.

### DETAILED DESCRIPTION OF THE PREFERRED EMBODIMENTS

A method of treating osteomyelitis of the jaws (OMJ) can include piezoelectric surgery to remove necrotic bone (sequestrectomy) and create bleeding spots (decortication). The OMJ can include secondary chronic OMJ caused by previous dental implant placements. The method can further include administering dual antibiotic therapy to the patient. The dual antibiotic therapy can include administering Augmentin (e.g., 1 g twice daily) and Metronidazole (e.g., 400 mg three times a day) to the patient for a period of about 3 weeks (e.g., 1 week before surgery and 2 weeks after surgery). Decortication improves blood supply from the deeper marrow spaces, surrounding soft tissues and periosteum, to the affected bone marrow. Inducing bleeding around the site can optimize a healing environment for osseous healing.

In piezoelectric bone surgery, micrometric ultrasonic piezoelectric vibrations are applied for cutting bone tissue. Piezoelectric ceramic contraction and expansion results in ultrasonic vibrations with a frequency ranging from about 25 to about 29 kHz. This is translated and amplified onto inserts resulting in linear micro-vibrations in a range of about 60 um to 200 um, exclusively cutting hard tissues and leaving soft tissues untouched. The bone surfaces can be irrigated using a peristaltic pump while the inserts vibrate linearly. High levels of energy, exceeding 5 W, are transferred to bone surfaces, permitting osteotomies even with highly mineralized bone. When compared to conventional surgical devices for managing OMJ, piezosurgical devices achieve reduced bacteria levels due to the disinfecting action of shock waves in the fluid environment and can achieve higher levels of control, precision, and safety.

Further, conventional tools for treating or managing OMJ include drills and/or microsaws which involves the use of a significant amount of pressure to remove the bone, can result in overheating and, ultimately, coagulative necrosis. Piezoelectric surgery, on the other hand, involves irrigation, gentle brushing pressure, and integrated saline spray to maintain a low temperature and visible surgical field. Unlike conventional methods for treating OMJ, the present method does not result in coagulative necrosis.

In an embodiment, the PIEZOSURGERY® device from MECTRON can be used, for performing the piezoelectric surgery. Inserts for the device can include, for example, sharp, smooth, and blunt inserts. Suitable coatings can include, e.g., diamond and titanium nitride. In an embodiment, sharp inserts coated with diamond can be used for the sequestromy and decortication. For removal of sharp and bony edges, smooth inserts also coated with diamond, can be used.

The present teachings are illustrated by the following examples.

### EXAMPLE

A sixty-six year old male patient presented to Kuwait University Dental Center for treatment regarding a non-healing wound in the right posterior mandible. The patient was in good health with no significant medical condition. The patient reported the following history relevant to the site of concern: missing mandibular anterior and right posterior teeth for more than 5 years. The only remaining tooth on the mandibular right quadrant was the canine. Anterior mandibular implants were placed 4 years ago and restored. Posterior right mandibular implants were placed and restored 3 years ago. 1 year after restoration, symptoms developed in the anterior region first, then very shortly (within weeks) in the posterior region all around the implant sites. Symptoms seem to be of inflammatory nature. Anterior implants were removed 2 years ago. Then shortly after, posterior implants were removed sequentially. The last implants to be removed were in the sites of the first molar and 2nd premolar (right mandible).

The patient reported having pain, swelling and pus discharge from the sites of implantation and removal since the last procedure. These symptoms were managed by several attempts of curettage and several antibiotic treatment courses. The antibiotics taken were mostly Amoxicillin 500 mg TID and Clindamycin (dose not available). Symptoms in the anterior mandible improved then gradually resolved. However, symptoms in the posterior mandible did not resolve and were a constant source of discomfort, swelling and pus discharge.

Upon clinical exam, no lymphadenopathy was detected during head and neck palpation. There was a relatively small hard mass that was palpated on the right buccal space region. Intra-orally, teeth were missing in the posterior right and anterior mandible with only canines remaining. Canines had significant recession and periodontal disease. An open wound was detected directly above the residual ridge around the 1st molar and 2nd premolar sites with pus discharge with a relatively large fistula. The site was painful upon buccal and lingual palpation.

Based on the history reported by the patient and clinical findings, a differential diagnosis of Chronic Secondary Osteomyelitis of the mandible was established. Diagnosis was later confirmed by radiologist, after requesting a CBCT, and an oral pathologist after biopsy.

The treatment plan included sequential debridement of the site, biopsy of soft and hard tissue, an antibiotic regimen of Augmentin (1 gram every 12 hours) and Metronidazole (400 mg every 8 hours for 7 days). The debridement included excising fistula, curettage of granulation tissue, sequestrectomy, and decortication under local anesthesia.

The site was irrigated with normal saline, followed by application of Eugenol impregnated alveolar dressing. Alveogyl, Septodont was administered to help with pain. A five day course of dual antibiotic (1 g Augmentin BID and Metronidazole 400 mg TID) was administered to minimize pus discharge and decrease chances of post-surgical infection. Augmentin was selected to cover β-lactam and broad spectrum bacteria. Metronidazole was selected to cover anaerobic bacteria. 0.12% Chlorohexidine mouth rinse and Catafam (50 mg PRN) were also prescribed.

After 1 week, the patient presented for surgical debridement. The patient reported improved symptoms. The site showed significantly less puss discharge and less sensitivity to palpation. Local anesthesia (a total of 2 cartridges 1.8 ml 2% lidocaine 1:100,000 epi), buccal and lingual infiltrations were used. Using a 15c blade, an incision was made from the residual ridge of the 2nd molar extending anteriorly to the right mandibular canine. A circumferential incision was made around the fistula which was removed, placed in a formaldehyde bottle and sent for biopsy. Full muco-periosteal flap was reflected after making a vertical releasing incision posteriorly. Blunt dissection was performed and the mental foramen and mental nerve were identified. The site was exposed and a large amount of granulation tissue was noted. All granulation tissue was curettaged and sent for biopsy in a formaldehyde bottle. Underlying bone was exposed and minimal intra-osseoss bleeding was observed. Using a piezosurgery unit with sharp inserts on cutting mode, necrotic bone was removed (sequestrectomy), bleeding sites were created (decortication), and bone specimen was collected and sent for biopsy in a formaldehyde bottle. Smooth inserts were then used to smoothen sharp edges. The buccal plate was smoothened using the smoothening inserts to remove sequestrum. The site was irrigated heavily with normal saline under the flap prior to closure. Residual bone chips were detected after irrigation. Eugenol impregnated alveolar dressing (Alveogyl, Septodont) was packed on the ridge and the flap was repositioned to the native site using multiple 4-0 PTFE sutures. A tension-free primary closure was achieved.

Augmentin (1 g every 12 hours) and Metronidazole (400 mg every 8 hours for 7 days) was prescribed. After one week, the patient reported significant improvement in symptoms. Intra-orally, the wound was still closed, with sutures intact, and no pus discharge was detected. Collagen deposits were detected indicating optimal wound healing. A small mass still palpated extra-orally (decreasing in size). Augmentin (1 gram every 12 hours) and Metronidazole (400 mg every 8 hours for 7 days) was re-prescribed.

After 2 weeks, the patient still reported no signs or symptoms related to the site, with no evidence of the small mass that palpated previously. Sutures were removed, the site was irrigated and the dual antibiotics were prescribed for 1 more week, totaling 3 weeks of dual antibiotics treatment (1 week before and 2 weeks after surgery).

The patient was recalled every 6 months, no signs and symptoms were detected or reported related to the site. A CBCT scan was requested after 28 months of surgery. All CBCT sans were evaluated by an Oral and Maxillofacial radiologist.

After 28 months, one cone beam computed tomography volume, measuring 140.25 mm in diameter by 51 mm in height, was acquired to evaluate mandibular right molar region for possible implant placement. A low-density area in the edentulous space of tooth 46 consistent with an extraction socket was identified, as seen in the previous study without the bony sequestrum. The buccal cortex was shorter than the lingual with small (less than 1 mm in diameter) area of higher (bony) density centered in the soft tissue. When compared to the previous study, the apical, mesial and distal to the buccal cortex onion-skin like areas of high density were completely opacified and could not be distinguished from the buccal cortex which is consistent with healing of the previously present reactive bone formation caused by the chronic infection seen in the previous study. The extraction socket with less than 1 mm bony sequestrum centered on the buccal soft-tissue window of the extraction socket appeared to be completely healed. Onion-skin like projections buccal to buccal cortex was no longer seen and apparent complete healing was observed. Complete healing of the previously present chronic osteomyelitis was observed.

It is to be understood that the methods described herein are not limited to the specific embodiments described above, but encompass any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A method of treating osteomyelitis of the jaws, comprising:
administering piezoelectric surgery to remove necrotic bone and create bleeding spots in an affected area of the patient's jaw; and
administering an antibiotic treatment to the patient for one week prior to the piezoelectric surgery and two weeks after the piezoelectric surgery, the antibiotic treatment including a first antibiotic targeting β-lactam and broad spectrum bacteria and a second antibiotic targeting anaerobic bacteria.

2. The method of treating osteomyelitis of the jaws as recited in claim 1, further comprising excising of fistula and curettage of granulation tissue prior to administering piezoelectric surgery.

3. The method of treating osteomyelitis of the jaws as recited in claim 1, wherein 1 gram of the first antibiotic is administered twice daily.

4. The method of treating osteomyelitis of the jaws as recited in claim 1, wherein 400 mg of the second antibiotic is administered three times a day.

5. The method of treating osteomyelitis of the jaws as recited in claim 1, wherein a piezosurgery unit with sharp inserts is used for the removal of necrotic bone and creating of bleeding spots.

6. The method of treating osteomyelitis of the jaws as recited in claim 5, wherein the sharp inserts comprise a diamond coating.

7. The method of treating osteomyelitis of the jaws as recited in claim 1, wherein the osteomyelitis of the jaws is caused by a prior dental implant and the affected area includes a site of the prior dental implant.

8. The method of treating osteomyelitis of the jaws as recited in claim 1, wherein the osteomyelitis of the jaws is secondary osteomyelitis of the jaws.

9. A method of treating osteomyelitis of the jaws, comprising:
excising of fistula and curettage of granulation tissue from an affected area of the patient's jaw;
administering piezoelectric surgery to remove necrotic bone and create bleeding spots in the affected area of the patient's jaw; and
administering an antibiotic treatment to the patient for one week prior to the piezoelectric bone surgery and two weeks after the piezoelectric surgery, the antibiotic treatment including a first antibiotic targeting β-lactam and broad spectrum bacteria and a second antibiotic targeting anaerobic bacteria.

10. The method of treating osteomyelitis of the jaws according to claim 9, wherein 1 gram of the first antibiotic is administered twice daily.

11. The method of treating osteomyelitis of the jaws as recited in claim 9, wherein 400 mg of the second antibiotic is administered three times a day.

12. The method of treating osteomyelitis of the jaws as recited in claim 9, wherein the osteomyelitis of the jaws is caused by a prior dental implant and the affected area is a site of the prior dental implant.

13. The method of treating osteomyelitis of the jaws as recited in claim 9, wherein the osteomyelitis of the jaws is secondary osteomyelitis of the jaws.

14. A method of treating osteomyelitis of the jaws, comprising:
administering piezoelectric surgery to remove necrotic bone and create bleeding spots in the affected area of the patient's jaw; and
administering an antibiotic treatment to the patient for one week prior to the piezoelectric bone surgery and two weeks after the piezoelectric surgery, the antibiotic treatment including administering a first antibiotic targeting β-lactam and broad spectrum bacteria twice daily and a second antibiotic targeting anaerobic bacteria three times a day,
wherein the osteomyelitis of the jaws is caused by a prior dental implant.

15. The method of treating osteomyelitis of the jaws as recited in claim 14, wherein the osteomyelitis of the jaws is secondary osteomyelitis of the jaws.
